(19) United States
(12) Patent Application Publication
(10) Pub. No.: US 2018/0036174 A1
(43) Pub. Date: Feb. 8, 2018

(54) SAFETY GLASSES

(71) Applicant: Christopher Randolph, Bergholz, OH (US)

(72) Inventor: Christopher Randolph, Bergholz, OH (US)

(21) Appl. No.: 15/598,556

(22) Filed: May 18, 2017

Related U.S. Application Data
(60) Provisional application No. 62/338,149, filed on May 18, 2016.

(51) Int. Cl. A61F 9/02

(52) U.S. Cl. CPC A61F 9/026

(57) ABSTRACT

A safety glasses adapter comprising a resilient frame having at least two apertures attached to one another at least two temple extensions extending outward from the at least two apertures, wherein at least one of the at least two temple extensions extends from each of the at least two apertures, and wherein a pair of glasses are reliably attached to the frame.

### CROSS-REFERENCE TO RELATED APPLICATION(S)

The present application claims priority to U.S. Provisional Patent Application No. 62/338,149 filed on May 18, 2016, entitled “Real Safe Safety Glasses” the entire disclosure of which is incorporated by reference herein.

### BACKGROUND OF THE INVENTION

### 1. Field of Invention

The present invention relates to the field of eyewear and safety accessories. More particularly to devices that adapt standard eyewear into safety glasses.

### 2. Description of Related Art

Eyes are delicate organs and subject to injury when contacted by foreign objects. Other forms of damage can be the result of over exposure to ultra violet light sources. To correct such exposure, someone may choose to wear sunglasses. Similarly, to protect against foreign objects, someone may selectively wear glasses during activities where eye damage may be more prevalent.

Aside from particular applications of glasses, some people require corrective lenses to improve or modify their vision. Regardless of the particular application or use of the glasses, they generally have a pair of lenses and temple extensions to engage the sides of the users head. While corrective lenses and sun glasses may provide protection against from foreign object, the lenses are generally positioned away from the user's face allowing for objects to enter the space between the lenses and the user's face.

Safety glasses are used to cover the void between the lenses and the user's face, but their style is limited. It is impractical for a user to carry multiple sets of glasses to be interchanged based on their specific use and protective capabilities.

Current solutions have provided for various elements added to glasses in an attempt to modify them for a wider range of protective uses. However, the current adapters are generally limited in their coverage and subject to becoming easily dislodged. Some adapters attach to the interior side of the lenses and attempt to cover the gap between the user's face and the lens. However, such an adapter is subject to falling out of place due to the limited attachment points.

Based on the foregoing, there is a need in the art a compact and effective adapter to modify any shape or style of glasses. A device that will proactively retain the glasses to the user's head and protect susceptible areas of the glasses from damage.

### SUMMARY OF THE INVENTION

A safety glasses adapter having a resilient frame having at least two apertures attached to one another, At least two temple extensions extend outward from the at least two apertures, wherein at least one of the at least two temple extensions extends from each of the at least two apertures,

In an embodiment, the at least two temple extensions extend from a peripheral edge of each of the at least two apertures.

In an embodiment, the adapter is molded to a shape of a pair of glasses.

In an embodiment, each of the two temple extensions comprise a plurality of attachment elements.

In an embodiment, the plurality of attachment elements retain temple extensions of a pair of glasses.

In an embodiment, each of the at least two apertures has an interior perimeter configured to engage an exterior perimeter of lenses.

In an embodiment, the adapter is molded after the adapter is heated.

In an embodiment, the at least two apertures are stretched over lenses of glasses.

In an embodiment, each of the at least two apertures have at least one sidewall extending around a perimeter of each aperture, wherein the at least one sidewall extends outward from an interior surface of the lenses of the glasses.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the safety glasses, according to an embodiment of the present invention;

FIG. 2 is a perspective view of the safety glasses, according to an embodiment of the present invention;

FIG. 3 is a perspective view of the safety glasses, according to an embodiment of the present invention;

FIG. 4 is a perspective view of the safety glasses, according to an embodiment of the present invention;

FIG. 5 is a perspective view of the safety glasses, according to an embodiment of the present invention; and

FIG. 6 is a perspective view of prior art, according to an embodiment of the present invention.

### DETAILED DESCRIPTION OF PREFERRED EMBODIMENTS

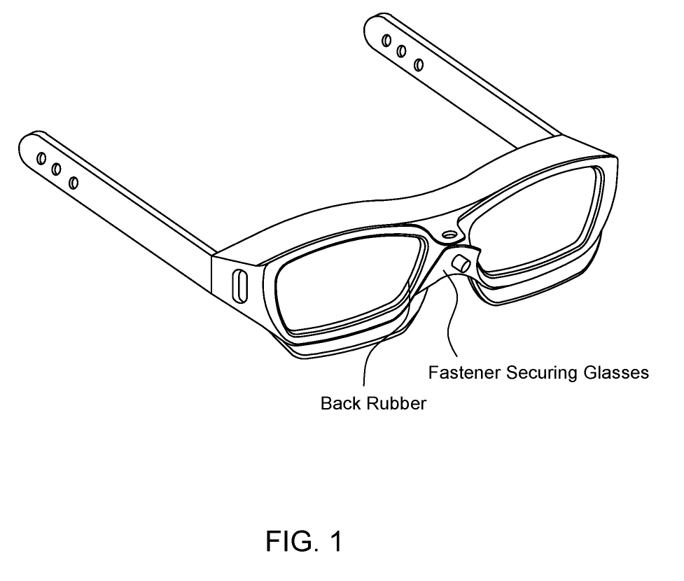

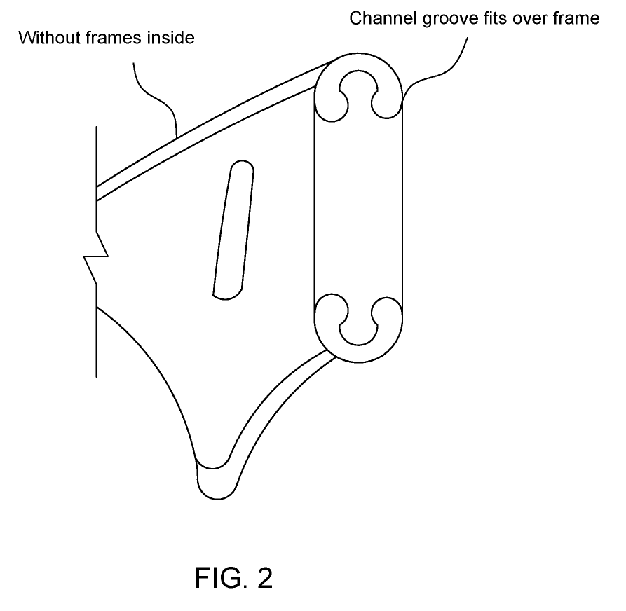

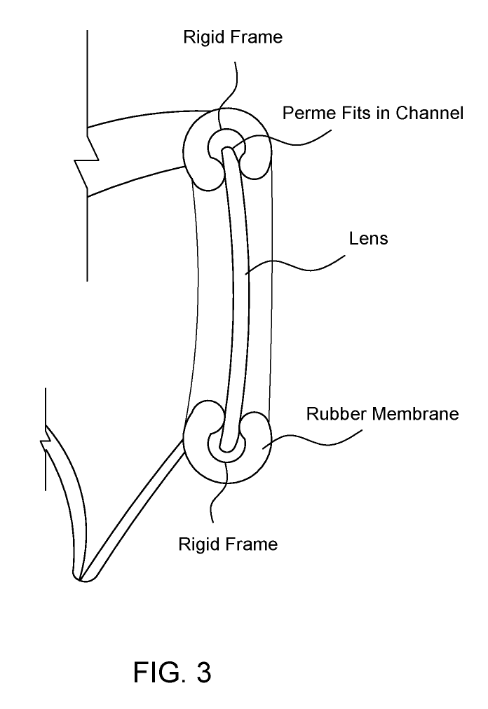

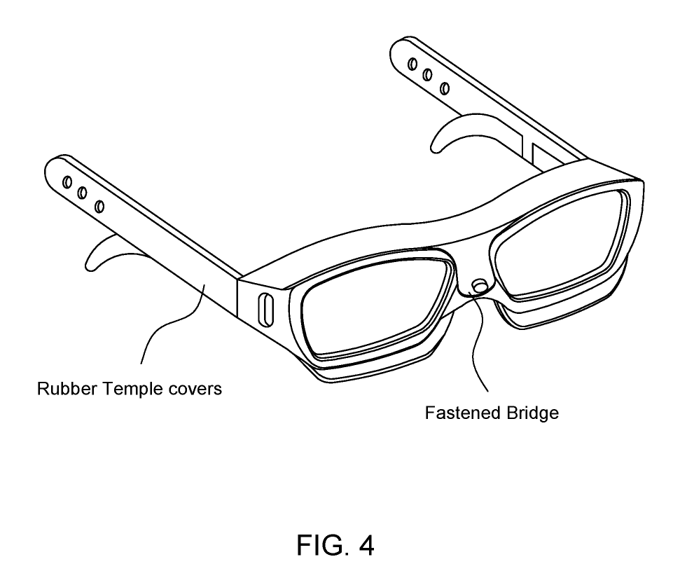

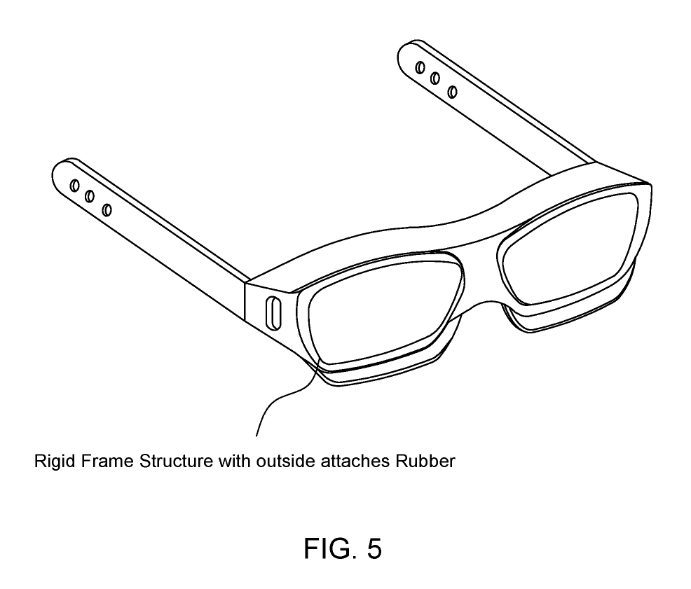

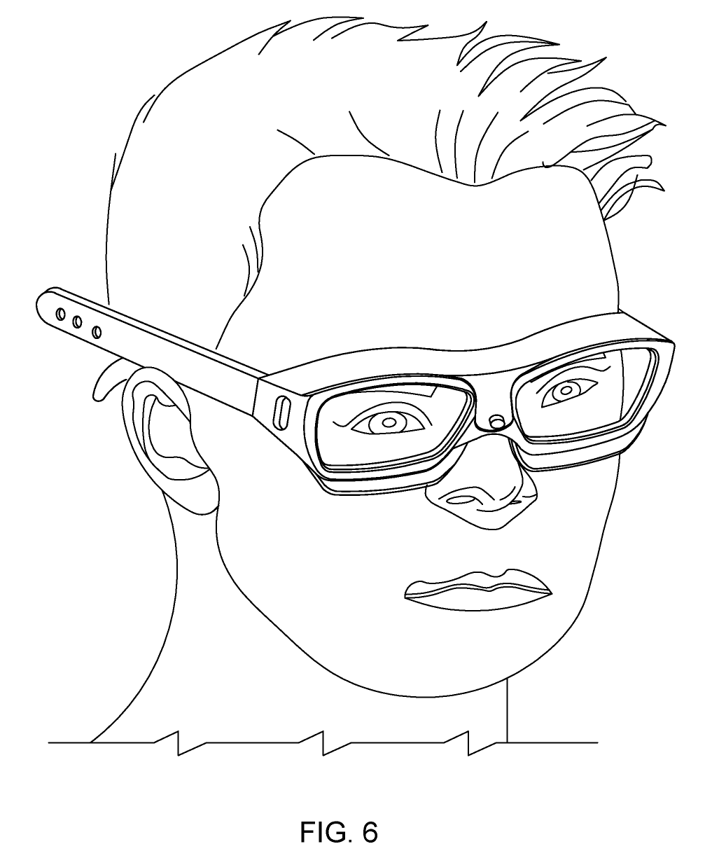

Preferred embodiments of the present invention and their advantages may be understood by referring to FIGS. 1-6, wherein like reference numerals refer to like elements.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

A safety glass system has a set of glasses and a releasably attached adapter device. The adapter is comprised of a resilient material and engages the set of glasses around a perimeter of each of the lenses and each temple extension. The adapter provides for at lease one sidewall extending away from an interior lens surface. The interior lens surface is proximal to the user's face, when the glasses are worn. The adapter sidewalls provide for a physical barrier against foreign objects from entering the space between the interior surface of the lens and the user's face.

The adapter has two apertures connected to one another. Each aperture is configured to receive the perimeter of a lens, from the pair of lenses. The adapter apertures may be expandable to allow for an adjustable aperture size. The adjustable aperture size allows for application of the adapter to various styles of glasses.

In an embodiment, the attachment of the adapter apertures is positioned between the two apertures. The adapter also has two temple extensions extending outward from a perimeter of each aperture opposite of the attachment point. In this configuration, the adapter components generally align with the structural elements of the glasses. The temple extensions of the adapter further provide attachment means.

In an embodiment, the attachment means may include loops, straps, clips, magnets, adhesive, or hook and loop fasteners. The attachment means is configured to retain a corresponding temple extension of the glasses.

A supplemental attachment means may be provided at the attachment point between the adapter apertures. The supplemental attachment means engages a bridge of the glasses. The bridge of the glasses separates the lenses.

In use, the adapter is installed onto a pair glasses. First, a first temple extension of the glasses is inserted into the corresponding temple extension of the adapter. This process is repeated on the opposite temple extensions. The attachment means actively engages the temple extension of the glasses. The tehmple extensions of the glasses are then slidably engaged to the temple extensions of the adapter such that the temple extensions of the glasses are slide into the adapter until the lenses align with the apertures of the adapter. The apertures of the adapter are then manipulated to engage the peripheral edge of the lenses. The sidewalls are positioned such that they cover the void between the glasses and the user's face.

In an alternative embodiment, the adapter is comprised of a rigid material and specifically adapted to accommodate a particular style of glasses. The attachment of the adapter to the glasses may be similar accommodating for the lack of flex in the rigid adapter.

In an alternative embodiment, the adapter is customizable to a tailored fit to a user's glasses. For example, the adapter may comprise a material that is reactive to heat. When the adapter is heated, the material becomes pliable and can be molded to a particular type of glasses. In use, the user would heat the adapter to a preset temperature until the molecules of the material were energized increasing the material pliability. The adapter is installed, as described to the set of glasses and the material is allowed to cool. As the material cools, the material contracts to a limited position based on the engagement of the pair of glasses. When the adapter has fully cooled, it can be removed from the glasses while retaining its customized molded shape.

In another embodiment, a pair of glasses are integrated into the adapter. In such an embodiment, the adapter and glasses are combined during manufacturing such that the adapter is permanently affixed to the glasses. The method of manufacturing the glasses includes at least the step of a pair of glasses having lenses being encases by the apertures of the adapter. An interior surface of the aperture receives an exterior perimeter of the lenses such that the lenses and the apertures are affixed to one another. The temple arms of the glasses are then in contact with the temple extensions of the adapter.

In another embodiment, the temple extensions of the adapter have a plurality of holes positioned along a length of each of the temple extensions. The plurality of holes facilitate a releasable engagement with either the temple arms of the glasses or a separate object such as a helmet or hat.

The interior surface of the apertures may have a channel or indentation extending inward towards a center of the adapter, which defines the aperture. The channel provides for positive interaction between the lens perimeter and the adapter aperture.

In another alternative embodiment, a front side of the adapter has a single aperture in a general oval shape. The single aperture is sufficient size to accept the pair of lenses of the glasses. While the single aperture may accept both lenses, in an embodiment, a clasp mechanism may bisect the single aperture, thereby forming two apertures from the single aperture. The clasp mechanism may provide further engagement with the bridge of the glasses, where the bridge of the glasses extends between the two lenses, central to the temple arms of the glasses.

In an alternative embodiment, the adapter further comprises a power source in electrical communication with a switch and a light source. The light source is general positioned on an outward surface of the adapter, where the outward surface is distal to the user's face. For example, a light source may be disposed within the adapter between the two apertures and/or on an outer perimeter of each adapter. The light source is configured to provide illumination in front of the user, when activated.

In another embodiment, the sidewalls may provide for one or more vents allowing airflow into the space between the lens and the user's eyes. The vents are configured to selectively allow the flow of air of fluids into the void thereby preventing condensation or excessive heat accumulation within the void.

In a particular embodiment, the adapter provides for a thin, lightweight, and extra stretchy rubber material, which can be molded to form and can easily attached to basically any pair of glasses. For example, regular glasses, prescription glasses, or safety glasses. The adapter ensures prevention of all debris, wind, water, splash guard, and, if necessary, light, from entering the eyes. Turning any pair of glasses in instant safety goggles.

In yet another embodiment, the apertures comprise a rigid or semi rigid interior insert to accept the perimeter of the lenses. The insert may provide for a channel having various geometric shapes to promote a positive engagement between the lenses and the insert, thereby attaching the adapter to the glasses.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A safety glasses adapter comprising:
a. resilient frame having at least two apertures attached to one another;
b. at least two temple extensions extending outward from the at least two apertures, wherein at least one of the at least two temple extensions extends from each of the at least two apertures, and wherein a pair of glasses are reliably attached to the frame.

2. The adapter of claim 1, wherein the at least two temple extensions extend from a peripheral edge of each of the at least two apertures.

3. The adapter of claim 1, wherein the adapter is molded to a shape of a pair of glasses.

4. The adapter of claim 1, wherein each of the two temple extensions comprise a plurality of attachment elements.

5. The adapter of claim 4, wherein the plurality of attachment elements retain temple extensions of a pair of glasses.

6. The adapter of claim 1, wherein each of the at least two apertures has an interior perimeter configured to engage an exterior perimeter of lenses.

7. The adapter of claim 3, wherein the adapter is molded after the adapter is heated.

8. The adapter of claim 1, wherein the at least two apertures are stretched over lenses of glasses.

9. The adapter of claim 1, wherein each of the at least two apertures have at least one sidewall extending around a perimeter of each aperture, wherein the at least one sidewall extends outward from an interior surface of the lenses of the glasses.
